(19) United States
(12) Patent Application Publication
(10) Pub. No.: US 2006/0014176 A1
(43) Pub. Date: Jan. 19, 2006

(54) Reactor for chemical synthesis

(76) Inventors: Radhakrishnan P. Iyer, Shrewsbury, MA (US); Seetharamaiyer Padmanabhan, Lexington, MA (US); John Edward Coughlin, Auburn, MA (US)

Correspondence Address: ELMORE PATENT LAW GROUP, PC, 209 MAIN STREET, N. CHELMSFORD, MA, 01863, US

(21) Appl. No.: 11/137,677

(22) Filed: May 25, 2005

Related U.S. Application Data
(60) Provisional application No. 60/574,465, filed on May 26, 2004.
(60) Provisional application No. 60/583,414, filed on Jun. 28, 2004.
(60) Provisional application No. 60/626,597, filed on Nov. 10, 2004.
(60) Provisional application No. 60/647,734, filed on Jan. 27, 2005.

(51) Int. Cl. C12Q 1/68; G01N 33/53; C12M 1/34; C07K 1/02; C07H 21/04

(52) U.S. Cl. 435/6; 435/7.1; 530/333; 536/25.3; 435/287.2

(57) ABSTRACT

The present invention relates to the design and use of a multipurpose reactor that may be adapted to accommodate a wide variety of solid phase and liquid phase reaction chemistries.

### RELATED APPLICATIONS

This application claims the benefit of U.S. Provisional Application No. 60/574,465, filed on May 26, 2004, U.S. Provisional Application No. 60/583,414, filed on Jun. 28, 2004, U.S. Provisional Application No. 60/626,597, filed on Nov. 10, 2004 and U.S. Provisional Application No. 60/647,734, filed on Jan. 27, 2005. The entire teachings of the above applications are incorporated herein by reference.

### GOVERNMENT SUPPORT

The Invention was supported, in whole, or in part by NIH Grant Number 5 UO1 A1058270-02.

### FIELD OF THE INVENTION

The present invention relates to a reactor for chemical synthesis.

### BACKGROUND OF THE INVENTION

Since the first report of solid-phase synthesis of peptides by Merrifield in 1962, several applications of using solid matrices have evolved over the past 50 years. For example, solid-phase synthesis is now routinely employed for the synthesis and manufacture of macromolecules such as peptides, carbohydrates, and di-, tri- or larger oligonucleotides.

Also, several organic reactions are routinely performed in solution phase employing reagents that are covalently bound to solid matrix. Following the reaction, the matrix containing the reagent is simply filtered off from the reaction medium enabling partial purification of the desired product from the solution. In another application, compounds attached to solid matrices that carry acidic and basic moieties are employed as “scavengers” of basic and acidic reagents respectively from reaction media.

Several catalysts employed in organic synthesis are often used as solid matrix. Compounds attached to solid matrices are also employed as sensors in detection devices. In yet another application, drug molecules attached to solid matrices are used as delivery systems for topical and systemic administration of drugs.

Macromolecules such as oligonucleotides as antisense compounds and as agents of RNA interference and peptides are synthesized and manufactured routinely using solid-phase synthesis. In this case, the first nucleotidic or amino acid residue (also referred to as the leader building block) is covalently attached to the solid matrix via a linker arm. The subsequent additions of monomeric units to the leader building block is carried out on the solid matrix. Upon completion of the assembly of the macromolecule, the matrix is treated with a reagent that cleaves the linker arm of the leader block thereby releasing the macromolecule into solution.

Many of these reactions are carried out using reagents that are sensitive to air and/or moisture. This is accomplished by operating the reactions in an inert atmosphere of argon or nitrogen in a closed reaction vessel such as a column. The column usually has a cylindrical configuration with the two ends of the cylinder carrying filters that hold the solid support within the body of the cylinder. The reactants are introduced into the column via inert gas pressure and the reaction takes place within the vessel between the leader monomer unit and the reactant.

In such multi-phase reactions where one or more components is a solid, liquid, or gas, proper contact between the phases is critical in ensuring efficient reactions that result in high yields. Often, an excess of reactants is required to be employed for ensuring that reactions go to completion. In addition, a continuous flow of inert gas needs to be maintained for achieving a state of “fluidization” so that intermixing of phases occurs. The excess reagents cannot be readily recovered resulting in wasteful processes and increased costs of a given operation.

Furthermore, since the reaction vessel is a closed assembly, access to the solid matrix during the reaction can only be made by opening the vessel and interrupting the reaction. Access to solid matrix is important because it will facilitate the determination of the physical changes of the matrix such as changes in swelling characteristics, particle size, pore size etc and allow for more efficient control of the reaction. In closed assemblies, the reactions can be monitored only by downstream sampling of the liquid exiting from the reactor. Indeed, for some synthetic steps, in a solid-phase reaction, it is preferable to directly analyze the solid matrix rather than relying exclusively on downstream sampling of the column effluent.

### SUMMARY OF THE INVENTION

The present invention overcomes the above-described drawbacks in the state of the art reactors for chemical synthesis. The present invention relates to the design and use of a multipurpose reactor that may be adapted to accommodate a wide variety of solid phase and liquid phase reaction chemistries.

### BRIEF DESCRIPTION OF THE DRAWINGS

The present invention will become more fully understood from the detailed description given herein below and the accompanying drawing which is given by way of illustration only, and thus, is not limitative to the present invention. In the following drawings, like reference characters designate the same or similar parts throughout the several views, and wherein:

FIG. 1 is a schematic diagram showing one example of the design of the reactor according to the invention.

FIG. 2 is a schematic diagram showing another example of the design of the reactor of the invention.

FIG. 3A is a schematic of one example of the design of the reactor of the invention adapted for use in size-exclusion chromatography.

FIG. 3B is a schematic of one example of the design of the reactor of the invention adapted for use in chromatography including but not limited to ion-exchange chromatography and affinity chromatography.

### DETAILED DESCRIPTION OF THE INVENTION

The multipurpose reactor of the invention has unique design features, and enables simultaneous and efficient mixing of solid, liquid and gas phases for a variety of applications in solid and/or liquid phase reaction chemistries.

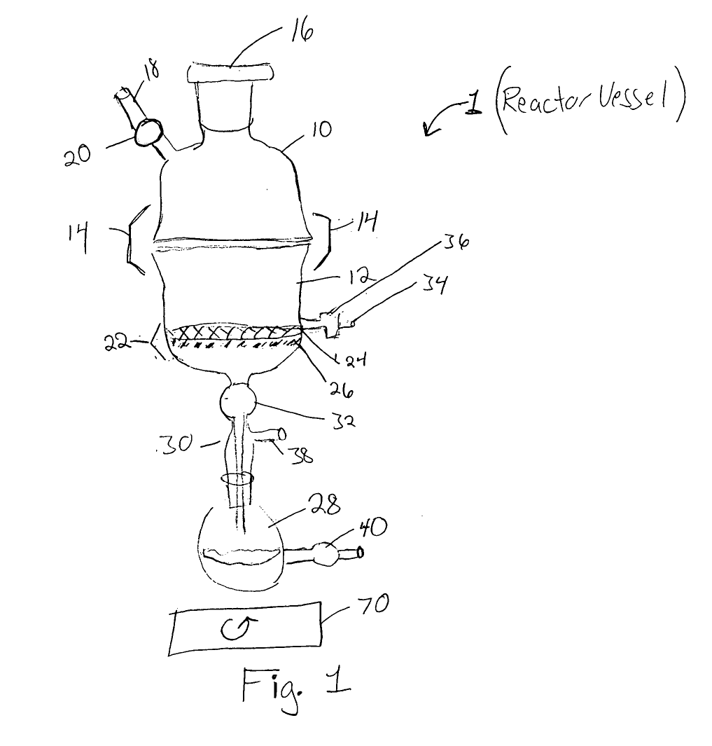

FIG. 1 shows a first embodiment for the design of a reactor that can be used for a variety of chemical synthetic applications. In this embodiment of the invention, the reactor comprises a reaction vessel 1 comprising an upper chamber 10 and a lower chamber 12 wherein the upper chamber is removably joined to the lower chamber and in solid/liquid/gas and gas contact with the lower chamber. For example, the upper and lower chambers may comprise flanged ends as shown in FIG. 1, wherein the flanged ends are held together by clamps 14. Alternate embodiments for removably securing the two chambers together are known in the art. For example, each chamber can be threaded so that the two halves can be screwed together. The reaction vessel 1 and its components can be derived from a variety of materials such as glass, polypropylene, Teflon, and glass-lined stainless steel.

The upper chamber further comprises a gas/liquid/solid inlet 16. The gas/fluid inlet 16 may optionally be designed to connect to another device in an airtight manner via a suitable adapter. For example, if the reaction vessel is a glass assembly, the opening can be a 24/40-glass joint that can fit into a 24/40 gas bubbling device. Alternatively, the opening can be fitted with septa through which inert gas or liquid or solid can be introduced into the reactor. Generally, the inlet 16 is used to introduce fresh chemical reactants into the reaction vessel and to admit pressurized inert gasses into the reaction chamber to facilitate chemical reactions while maintaining an airtight inert gas atmosphere in the reaction vessel 1.

Pressure relief means are generally associated with the upper chamber 10 or the lower chamber 12 of the reaction vessel. Preferably, pressure relief means 18 are associated with the upper chamber as is shown in FIG. 1. Pressure relief means 18 may be a pressure relief outlet port as is shown in FIG. 1, comprising a pressure relief switch means 20 for opening and closing the port as necessary to relieve pressure in the reaction vessel. The pressure relief switch means 20 as well as all switch means described herein may be of any convenient design compatible with the subject methods and devices. For example, inlet and outlet ports of the invention may be opened and closed with commercially available Teflon® diaphragm valves.

The lower chamber 12 is equipped with a filter unit 22 which comprises a filter 24 which may be a membrane filter, a polypropylene mesh, or a frit (of suitable pore size) and a filter support means 26 which may be a mesh, grid or other support that may be fixed to the wall of the lower chamber 12 and is capable of supporting the filter 24 and a solid-phase or solution-phase synthesis of a chemical above the filter 24 while allowing reagents to pass below the filter unit 22. Alternatively, the filter unit 22 can be designed to be a false bottom unit integrated into a “pull away” or “pull down” module, or alternatively a diaphragm, which can be opened or closed as needed. These latter designs allow the solid matrix to be washed out of the reaction vessel and facilitate more efficient cleaning of the vessel.

In the case of solid-phase synthesis, a solid matrix used in the reaction can be placed on top of the filter 24 and the filter unit 22. The chemical product assembly occurs on the solid matrix via a series of step-wise reaction sequences involving reactants added to the reaction vessel at appropriate times, preferably through the inlet 16 of the upper chamber 10. At the end of the reaction, the solid matrix can be removed by separating the upper chamber 10 and the lower chamber 12 and accessing the lower chamber for removal of the solid matrix. The chemical product can then be isolated from the solid matrix. Alternatively, the use of false bottom or diaphragm design as the filter unit 22, allows the matrix to be emptied as slurry into a collection reservoir 28 in solid/liquid/gas and gas contact with the lower chamber as shown in FIG. 1.

The lower chamber 12, may further comprise an adapter 30 for providing an airtight connection to the collection reservoir 28, and the adapter 30 may further comprise a collection reservoir switch means 32 for preventing or allowing solid/liquid/gas to enter the collection reservoir 28 from the lower chamber 12. The adapter 30 may also further comprise a gas port 38 suitable for connecting to a vacuum line or other means for creating a draw in the lower chamber 12 to draw reactants and excess reagents through the lower chamber 12 from above the filter means 22 to below the filter means 22 and into the collection reservoir 28 as desired.

The collection reservoir may further comprise a collection reservoir port 40 that may be used for drawing reactants and excess reagents into the reservoir or for collecting the contents of the reservoir 28.

The lower chamber 12 optionally comprises a sampling port 34 that allows a sample of solid matrix to be drawn for analysis during the chemical reaction. Liquid reactants can also be sampled from port 34. The sampling port 34 may comprise a sampling port switch means 36 capable of being opened and closed to allow access to the chemical reaction mixture in the lower chamber 12.

Optionally, the entire reaction vessel shown in FIG. 1 including the collection reservoir 28 can be mounted on a shaker assembly such as an orbital shaker 70 to ensure thorough mixing of the reactants during the reaction. The shaker assembly is useful during solid-phase synthesis when a physical mixing means such as a stir bar or mechanical device within the reaction chamber is often required. By mounting the reaction vessel 1 on a shaker assembly, the need for a separate stirring or mixing device within the reaction chamber is eliminated and the shaker unit provides the kinetic energy required for any such reaction to cause appropriate mixing of the reaction phases.

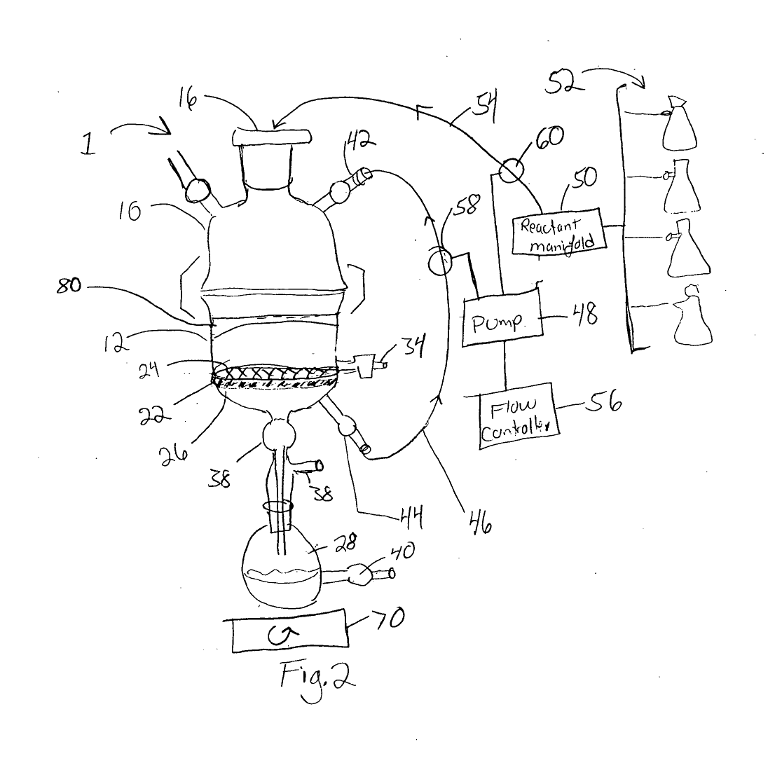

FIG. 2 shows another embodiment of the invention. This embodiment includes a reaction vessel 1 having all of the features described above and shown in FIG. 1 and also includes additional features to form an integrated chemical reactor system as shown in FIG. 2 and as will be described below.

In one aspect of the invention as shown in FIG. 2, the upper chamber 10 further comprises a recycled reagent inlet port 42 adapted for connection to a conduit means 46 such as tubing. The lower chamber 12 further comprises a recycled reagent outlet port 44 adapted for connection to the conduit means 46. The recycled reagent outlet port 44 of the lower chamber 12 can be connected to the recycled reagent inlet port 42 of the upper chamber 10 via conduit means 46 connected through a pump 48 via a pump head 58. In turn, the pump 48 is connected with pump head 60 via a reactants manifold 50 connected with one or more reactant reservoirs 52 that hold the chemical reactants to be added to the reaction vessel via reactant conduit means 54.

The pump 48 has at least two pump heads 58 and 60 that serve two functions (a) it helps draw the reactants reservoir 52 contents into the reaction vessel via the reactant conduit means 54 connected through the gas/solid/liquid/gas inlet port 16 via pump head 60 and (b) it helps in recycling the contents of the reaction vessel 1 through recycled reagent ports 44 and 42 via pump head 58. The pump 48 may optionally be connected to a flow controller 56 that may be used to modulate the delivery of reagent or the rate of recycling throughout the reaction vessel in order to maximize chemical reactions occurring in the reaction vessel. The concept of recycling the liquid phases in the vessel serves multiple objectives including helping to ensure efficient intermixing and contact between the liquid, solid, and gaseous phase for fast and efficient reactions, and helping to minimize reagent consumption in the reaction. The recycling can be done in both forward, as well as, reverse directions, at different speeds. Forward recycling generates turbulent forces that help in the mixing of solid and liquid phases thereby aiding mixing. The combined fluid actives of forward and reverse recycling facilitates thorough mixing of the solid and liquid reaction phases thereby resulting in rapid reaction kinetics and highly efficient synthesis without causing mechanical abrasion of solid support.

The pump 48 is preferably a peristaltic pump but can also be a vacuum pump or any other pump suitable for use with the subject devices and methods of the present invention.

The entire chemical reaction process incorporating the reactor of the invention can be automated as known in the art. For example, a programmable electronic control unit may be added to the reactor system shown in FIG. 2 to conduct the automated operations of the system including but not limited to, regulating the pump 48 activity flow rate and pumping activity, dispensing the reactants from the reactant reservoirs 52 into the reaction vessel 1, and monitoring the reaction by sampling the chemical reaction via the sampling port 34 that may be further interfaced with an electronic testing device capable of giving information about the progress of the chemical reaction. Pneumatic systems may also be incorporated for better control of fluid flow while maintaining an inert atmosphere. For example, pneumatic valves for delivery of reagents using solenoid valves may be used which upon activation, forces pressurized gas into the solvent and reactant chambers thus allowing liquids to enter the reactor. The solenoid valves may be operated by AC current or by analog to digital converters that will then be automatically controlled via a programmable electronic unit or integrated into a pre-existing electronic platform. This will create a fully automated reactor system.

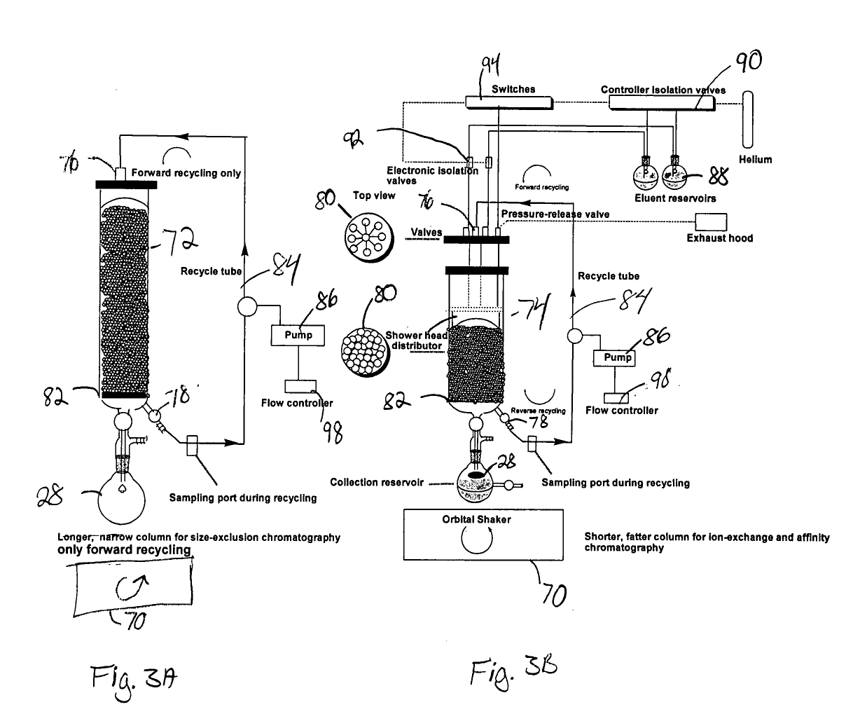

The reaction vessel of the invention may also be adapted with a “shower head distributor” (SHD) 80 (shown in FIGS. 2 and 3) for uniform distribution of reagents on the sold matrix during recycling and reagent feeds. The SHD may be adjustably positioned in within the reactor in any convenient location such that during solid phase synthesis, as the reagent liquid enters the SHD whether via direct feed or recycle feed, the reagent will be sprayed into the support uniformly.

The reactor of the invention may be adapted to accommodate a wide variety of solid phase, liquid phase and combination solid/liquid phase reaction chemistries. Examples of such chemistries include, but are not limited to: large-scale purification of recombinant proteins, monoclonal antibodies, and their conjugates using affinity chromatography, ion-exchange chromatography, and size-exclusion chromatography; manufacture of nucleic acids, peptides, and carbohydrates by solid-phase methodologies; solid-, and solution-phase combinatorial synthesis of small molecules for drug discovery, as well as, manufacture of small molecules; preparation of functionalized solid matrices such as beads, films, and pins carrying different types of ligands, such functionalized solid matrices find extensive use as (a) sensors in detection devices, (b) natural product extractions, (c) scavenging ligands in environmental clean up of toxic materials, (d) biomedical media for Radio-immuno assays, Fluorescent immuno-assays, ELISA, affinity chromatography and size exclusion chromatography; and (e) Drug delivery systems for topical and systemic administration of drugs.

In one embodiment, the reaction vessel 1 contains a solid phase polymer resin dispensed on the filter unit 22 of the lower chamber 12, preferably a solid phase peptide synthesis resin, such as crossed linked polystyrene. In some embodiments, the vessel is adapted to large scale synthesis, and/or contains at least 10, preferably at least 100, more preferably at least 1,000, more preferably at least 10,000 g resin (dry weight). Such embodiments are capable of simultaneously activating and coupling 100 g amino acid per step and provide yields of from 100 to 5,000 g of protected decapeptides. Solid phase polymer synthesis reaction chemistries are widely known in the art. For example, a typical peptide synthesis is conducted by the following procedure: The N-terminus of the resin-bound amino acid, peptide (protected by FMOC) is deblocked in a solution of piperidine in dimethyl formamide (DMF), for example. The next amino acid in the sequence is coupled to the resin-bound peptide with coupling agent such as dicyclohexylcarbodiimide (DCC), 2-(1H-benzotriazole-1-yl)-1,1,3,3-tetramethyluronium tetrafluoroborate (TBTU), 2-(1H-benzotriazole-1-yl)-1,1,3,3-tetramethyluronium hexaflurophosphate (HBTU) in a solution of DMF, for example. An activating agent such as 1-hydroxybenzotrazole (HOBt) may be used to improve rate and selectivity of the coupling reaction and to decrease racemization. The unreacted amino acid, reagents, and by-products are removed from the resin by washing and filtration. The washing and filtration process is then repeated. The N-terminus of the peptide is then deblocked; another amino acid is added to the chain, the system is then washed and filtered, etc. The process is repeated until all the desired amino acids have been added to the peptide chain in the desired order. The remaining blocking groups are then removed from the peptide, the peptide is cleaved from the resin, and the peptide is collected.

In another embodiment, the reaction vessel 1 contains a solid phase support dispensed on the filter unit 22 of the lower chamber 12, preferably a solid phase synthesis resin, such as crossed-linked polystyrene, or controlled-pore-glass (CPG) linked to a leader nucleoside. In more particular embodiments, the vessel is adapted to large scale synthesis, and/or contains at least 10, preferably at least 100, more preferably at least 1,000, more preferably at least 10,000 g of support (dry weight). Such embodiments are capable of simultaneously activating and coupling 100 g nucleoside phosphoramidites or H-phosphonates per step and provide yields from 100 to 5,000 g of polynucleotides following the synthesis cycle steps consisting of detritylation, coupling, capping and oxidation or sulfurization.

Such solid phase polymer synthesis reaction chemistries for the synthesis of polynucleotides are widely known in the art. For example, a typical polynucleotide synthesis is conducted by the following procedure: The 5′-O-4,4-dimethoxytrityl (DMT) group of the support-bound nucleoside is deblocked in a solution of dichloroacetic acid in dichloromethane (DCM), for example. The next nucleotide in the sequence is coupled to the support-bound nucleoside via the corresponding nucleoside 5′-O-DMT-3′-O-β-betacyanoethyl-phosphoramidite using a coupling agent such as 1H-tetrazole, ethylthiotetrazole, 4,5-dicyanoimidazole, or benzimidazolium trifluromethane sulfonate etc. The unreacted phosphoramidites, reagents, and by-products are removed from the support by washing and filtration. The washing and filtration process is then repeated. The resulting internucleotidic phosphite is then oxidized (iodine/pyridine/water) or sulfurized (3H-benzodithiole-3-one-1,1-dioxide) resulting in the formation of phosphotriester or thiophosphotriester linkage. The 5′-O-DMT-terminus of the coupled product is then deblocked, another nucleoside phosphoramidite is added to the chain, and the synthetic sequence repeated until all the desired nucleoside units have been added to the polynucleotide chain in the desired order.

Following the assembly of the polynucleotide, the support is removed from the reactor vessel and deprotection and cleavage reactions are carried out to obtain the polynucleotide free in solution. In a typical protocol for deprotection, the solid support containing polynucleotide is treated with aq. NH₄OH and heated at 55° C. for 12 h. The deprotection is monitored by analytical HPLC and after completion of deprotection, the solid support is filtered, washed with water and the aqueous solution is acidified with acetic acid to pH 6.5-7.0. The crude compound is purified by preparative HPLC to isolate pure polynucleotide directly as its ammonium salt. Phosphorothioate polynucleotide typically has ³¹P-NMR, δ 55-56 ppm.

In an another aspect of the invention, the reactor of the invention as exemplified in FIGS. 1 and 2 may be used in the functionalization and loading of various solid supports. In one embodiment of this aspect of the invention, efficient loading of nucleosides on succinylated-CPG using the reactor of the invention in conjunction with, for example, N.Ndimethylformamide (DMF) as a solvent for facilitating recovery of excess nucleoside results in high economy of operations. Without being limited to any scientific theory, it is believed that in order to have efficient loading of functionalized solid supports in the multi-phase reactions necessary to accomplish such loading, it is necessary to have effective mixing and contact of the functionalized solid support with the nucleoside and various reagents. Additionally, in order to minimize exposure to toxic solvents and air-borne support particles, it is important to perform all operations in a closed system. The reactor of the present invention meets all of these requirements. The reactor of the invention incorporates various elements to facilitate solid-liquid mixing, for example, the reactor of the invention provides, a) orbital shaking of the solid and liquid phases, (b) fluidization of phases induced by inert gas, and (c) active recycling of the liquid phase. The reactor of the invention as shown in FIG. 2 provides both recycling and orbital shaking which can be optionally and independently controlled. Since all operations using the reactor of the invention are performed in a closed system, human exposure to reagents and supports are minimized while maintaining the necessary anhydrous conditions in an inert atmosphere.

In yet another embodiment, the reactor shown in FIGS. 1 and 2 may be used in chromatographic purification of synthetic as well as biologically derived macromolecules such as recombinant proteins and antibodies using different purification technologies such as reversed-phase HPLC, affinity chromatography, ion-exchange chromatography, and size-exclusion chromatography. Alternatively the reaction vessel 1 of FIGS. 1 and 2 may be replaced with a reaction vessel 72 of FIG. 3A or the reaction vessel 74 of FIG. 3B having a cylindrical shape preferred for various types of chromatography. Such cylindrical reaction vessel may comprise a single, cylindrical chamber 74 of FIG. 3B or cylindrical column 74 as shown in FIG. 3B, capable of being loaded with a chromatography matrix. It is preferable that for use in size exclusion chromatographic purification, a reaction vessel with a cylindrical configuration, but with a longer narrow column 74 as shown in FIG. 3B is preferred for increased HETP.

FIGS. 3A and 3B exemplify reactors of the invention adapted for chromatography. The reactors shown in FIGS. 3A and 3B comprise a cylindrical chamber 72 or 74 comprising at least one solid/liquid/gas inlet 76 and at least one solid/gas/liquid outlet 78; a filter unit 82; disposed in said cylindrical chamber 72 or 74 capable of supporting chromatography material while allowing liquid phase reagents to pass below the filter; a collection reservoir 28 associated with the cylindrical chamber and in solid/liquid/gas contact with the cylindrical below the filter; and wherein the at least one solid/liquid/gas inlet 76 and the at least one solid/liquid/gas outlet 78 are connected via a conduit means 84 wherein said conduit means 84 is connected to recycling means, preferably a pump 84 capable of causing the recycling of material between the at least one solid/liquid/gas inlet 76 and the at least one solid/liquid gas outlet 78.

As shown in FIG. 3B, the reactor may be further comprise eluent reservoirs 88 under the control of controller isolation valves 90, electronic isolation valves 92 and appropriate switches 94. The pump 86 may be under the control of a flow controller 98. A top view and a bottom view of the SHD 80 useful in all embodiments of the invention is shown in FIG. 3B as well.

As opposed to the current technology of static-bed chromatography, in the case of the reactor of the invention shown in FIGS. 1, 2 and 3A-3B, the concept of orbital shaking coupled with active recycling is expected to improve chromatographic efficiency in a number of ways: (a) provide greater height equivalent to a theoretical plate (HETP) for better chromatographic resolution of molecules; (b) result in efficient intermixing and contact of the chromatographic media (e.g., affinity and size exclusion chromatography) with the protein, in the biological medium; (c) allow more efficient utilization of chromatographic media compared to the currently used chromatographic procedures and instrumentation which are mainly based on static bed column with single pass technologies; and (d) enable maximum recovery of the target compound from the medium resulting in more economical processes.

Additionally, the following features of the reactor of the invention will help advance different modalities in the practice of chromatography.

For affinity chromatography and ion exchange chromatography:
a) orbital shaking will facilitate uniform packing of the column, and initial washing steps during packing of the column;
b) active recycling (forward and reverse) of liquid phase will ensure that the target protein or antibody is fully contacted and captured by the solid support with minimal loss in flow-through;
c) both recycling and orbital shaking will be optionally, and independently controlled depending on the stage of chromatographic purification;
d) the recycling ports allow a sample to be withdrawn periodically for analysis to ensure complete capture of the target protein by the support. It can also be used for eluent collection.
e) the SHD will prevent channeling and more efficient separation of the target molecule in ion-exchange process;
f) in ion-exchange chromatography, superior separations can be expected based upon increased HETP;
g) allows the use of lower ratio of chromatographic media to the product; and
h) universally applicable; can be implemented using different types of commercially available chromatographic media, for example media can be used for expanded bed chromatography.

For size-exclusion chromatography: only forward recycling will be used to help increase height equivalent to a theoretical plate (HETP) and facilitate separation between proteins of very close molecular weight. This strategy will ensure that during recycling, the fast-moving protein band of high molecular weight is not allowed to go back to the column but is collected via sampling port as it reaches the bottom of the column. Different approaches may be used to ascertain elution of the protein: (a) using dyes of different molecular weights that serve as positional molecular weight markers, and (b) by employing a UV detection for the eluent. It is possible that in certain circumstances a cylindrical column configuration such as that shown in FIG. 3A, may render orbital shaking ineffective in size-exclusion chromatography. However, forward recycling will enable increased HETP and more effective contact between solid-liquid interphase.

The design features of the reactor of the invention adapted for use in chromatography make it a versatile system for conducting large-scale purifications of biomolecules using chromatographic methods. Example 4 describes the use of the reactor of the invention for the isolation and purification of a recombinant proteint, OP-1 (a bone morphogenic protein) from cell media.

### EXAMPLES

### Example 1

### Loading of Nucleosides on a Solid Support

As a representative example, we carried out the loading of 5′-O-4,4-dimethoxytriyl-N-benzoyl-deoxyadenosine (DMT-N^{Bz}dA) and 5′-O-4,4-dimethoxytrityl thymidine (DMT-T) on succinylated CPG, using the reactor of the invention in conjunction with anhydrous DMF as solvent. Using 100 g supports in each batch, we were able to achieve high nucleoside loading of 70 to 80 μmol/g with only three equivalents of nucleoside within 12 to 14 hours. Both filtration and drying operations were performed in LOTUS. The nucleoside-loaded CPG was then treated with CAP A and CAP B in the reactor. Following orbital shaking coupled with active recycling, the reaction mixture was filtered, washed and dried. Thus, the nucleoside loading, as well as, the attendant process operations could be conveniently performed in reactor of the invention. Additionally, the use of DMF instead of pyridine prevented any unpleasant odor and also aided the facile recovery of excess nucleoside, which is especially advantageous while using expensive nucleosides. Indeed, the recovery of excess nucleoside was carried out by simple aqueous work-up of the DMF filtrate. Subsequently, the recovered nucleosides (DMT-N^{Bz}dA) were successfully loaded onto succinylated CPG. We believe that efficient intermixing of phases, induced by orbital shaking coupled with active recycling, along with on-line monitoring, is the key to faster reactions and dramatically improved nucleoside loading on solid support using the reactor of the invention.

It is pertinent to mention that nucleoside loading on succinylated CPG could also be carried out by simple orbital shaking using DMF as a solvent. Thus, we carried out loading on succinylated CPG (100 g batches) using five equivalents of DMT-N^{Bz}dA and DMT-T. Using this procedure, nucleoside-loaded CPG with loadings ranging from 60 to 70 micromol/g could be obtained. Using similar procedures, we have also carried out nucleoside loadings on carboxy-terminated wide-pore silica, Tentagel, and aminomethyl polystyrene.

Materials. DMT-N^{Bz}dA and DMT-T were obtained from Reliable Biopharmaceuticals (Mo.) and used as such. Anhydrous pyridine and dimethylformamide from reputed vendors were freshly distilled from CaH₂ prior to use. CPG was obtained from Prime Synthesis (Pa.). Other reagents such as EDC, N,N-Dimethylaminopyridine (DMAP), and triethylamine were obtained from reputed vendors and used as such. Cap A and Cap B were obtained from American International Chemicals (Mass.).

### Example 2

### Synthesis of Dinucleotide

We tested the use of the reactor of the invention in the solid-phase synthesis of a dinucleotide 5′-U_{2′-OMe} dA3′ using the synthesis protocol described previously. The HPLC profile of the crude showed (data not shown) less than 2% unreacted dA nucleoside where as that prepared using an Expedite synthesizer gave variable amounts of unreacted dA in synthesis. Considering that 2′-_{OMe}U phosphoramidite is a hindered nucleoside, the high efficiency synthesis of the dinucleotide using the reactor of the invention represents a very significant step in solid phase synthesis, particularly of oligonucleotides. Excess phophoramidite was recovered and CPG was recycled after functionalization.

### Example 3

### Chromatographic Purification of Crude Dinucleotide

In order to demonstrate the utility of the reactor of the invention and the concept of orbital shaking coupled with active recycling in purification of molecules, we purified the crude dinucleotide of Example 2. For this purpose, the commercially available Bonda Pak C-18 resin (33 g) was loaded in the reactor. By using the orbital shaking coupled with active recycling concept, the material was packed in the reactor chamber by washing with a few column volumes (2×200 mL) of water. The crude (5 millimol) was loaded on to the column. Elution was carried out with water followed by water/acetonitrile, 95/5; water/acetonitrile, 85/15; and water/acetonitrile 70/30 and 100% CH₃CNeach 200 mL volume. Each eluent was collected via the recycling port, and a sample analyzed by reversed-phase HPLC (data not shown).

### Example 4

### Large Scale Purification of Biomolecules

A number of initial experiments will be carried out to optimize parameters such as rpm of shaker, recycle flow rate, and volume, bed volume to weight of crude protein. For expanded bed, ion-exchange chromatography, four to five commercially available rainbow molecular weight markers in the Mᵣ range of 10000 to 250000 may be used. To determine optimal resin to OP-1 protein ratio, adsorption isotherm experiments will be performed as described⁸. Briefly, protein solution will be prepared in 10 mM sodium acetate buffer (pH 7.0, 2M NaCl) filtered through 0.45 um membrane filter. Equal volume of different concentration of buffered protein solution will be added to a series of flasks containing 1:5 v/v suspension of the resin. The flasks will be incubated by shaking for 2 h at 25° C. (120 rpm) for equilibrium to be established. Aliquots of the solution will be filtered through a 0.45 um filter and protein concentration of supernatant determined using UV at 280 nM. Amount of bound protein will be determined by mass balance. For size exclusion chromatography, a dye kit will be used of blue Dextran 2000 (MW, 2,000,000), Yellow Dextran (20,000) and vitamin B₁₂ molecular weight of 1,357) (both available with Amersham Biosciences).

The following steps are exemplary of the purification protocol that will be followed for OP-1 using the reactor of the invention as shown in any one of FIGS. 1-2, which is much simplified compared to traditional methods of microfiltration, centrifugation, and chromatography.

Step 1. Pack the reactor column uniformly with ion exchange resin (Expanded bed chromatography) using orbital shaking coupled with active recycling.

Step 2. Introduce the cell media containing the target protein OP-1, through the bottom port using recycle pump and recycle the media through the bed.

Step 3. Carry out elution of the protein employing orbital shaking coupled with active recycling as desired.

Step 4. Monitor eluent by UV and combine fractions using Western blot

Step 5. Combine OP-1 fractions, desalt by membrane filtration

Step 6. Carry out size exclusion chromatography using LOTUS: (a) Introduce the OP-1 fractions from step 5 tagged with marker dyes into LOTUS packed with resin. (b) Perform forward recycle. (c) Carry out elution, collect fractions by UV monitoring, combine fractions using western blot. (d) Lyophilize OP-1 fractions to obtain product.

The OP-1 thus obtained will be evaluated in a functional assay. The results (yield, and purity) of this simplified isolation and purification protocol will be compared with that currently used for OP-1

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A reaction vessel for chemical synthesis comprising:
an upper chamber and a lower chamber, said upper chamber being removably joined to said lower chamber and in solid/liquid/gas contact with said lower chamber, said upper chamber comprising a liquid/solid/gas inlet;
pressure relief means associated with at least one of the upper or lower chambers;
a filter unit disposed in said lower chamber capable of supporting solid or solution phase synthesis of a chemical above the filter while allowing excess reagents to pass below filter;
a collection reservoir associated with the lower chamber and in solid/liquid/gas contact with the lower chamber below the filter; and
switch means for preventing or allowing solid/liquid/gas contact between the lower chamber and the collection reservoir.

2. The reaction vessel of claim 1, further comprising a sampling port in solid/liquid/gas contact with the lower chamber above the filter unit.

3. The reaction vessel of claim 1, further comprising a recycled reagent inlet port associated with the upper chamber and a recycled reagent outlet port associated with the lower chamber below the filter unit, wherein the recycled reagent inlet port and the recycled reagent outlet port are in solid/liquid/gas contact via a conduit means.

4. The reaction vessel of claim 3 further comprising a pump in pressurized contact with the conduit means, wherein said pump is capable of pumping excess reactants from the lower chamber into the upper chamber via the conduit means.

5. The reaction vessel of claim 4 further comprising a reactants manifold in pressurized contact with the pump, wherein the reactants manifold is further in solid/liquid/gas contact with the at least one reactants reservoir and the liquid/solid/gas inlet of the upper chamber via a reactant conduit means.

6. The reaction vessel of claim 1 wherein the reaction vessel is mounted on a shaker assembly.

7. A method of solid-phase oligonucleotide synthesis comprising the steps of:
a) providing the reaction vessel of claim 1;
b) placing a resin-bound nucleotide or modified nucleotide on the filter unit in the lower chamber of the reaction vessel;
c) conducting stepwise oligonucleotide synthesis of an oligonucleotide within the reaction vessel; and
d) isolating the oligonucleotide from the resin.

8. A method of solid phase peptide synthesis comprising the steps of:
a) providing the reaction vessel of claim 1;
b) placing a resin-bound peptide or modified peptide on the filter unit in the lower chamber of the reaction vessel;
c) conducting stepwise synthesis of the peptide within the reaction vessel; and
d) isolating the peptide.

9. A method of solid-phase synthesis comprising the steps of:
a) providing the reaction vessel of claim 1;
b) placing a resin-bound reagent on the filter unit in the lower chamber of the reaction vessel;
c) conducting a reaction of the resin-bound reagent with a another reactant within the reaction vessel; and
d) isolating the product.

10. An oligonucleotide prepared in accordance with the method of claim 7.

11. A peptide prepared in accordance with the method of claim 8.

12. A multipurpose reaction vessel comprising:
an upper chamber and a lower chamber, said upper chamber being removably joined to said lower chamber and in solid/liquid/gas contact with said lower chamber, said upper chamber comprising a liquid/solid/gas inlet;
pressure relief means associated with at least one of the upper or lower chambers;
a filter unit disposed in said lower chamber capable of supporting solid or solution phase synthesis of a chemical above the filter while allowing excess reagents to pass below filter;
a collection reservoir associated with the lower chamber and in solid/liquid/gas contact with the lower chamber below the filter; and
switch means for preventing or allowing solid/liquid/gas contact between the lower chamber and the collection reservoir.

13. The multipurpose reaction vessel of claim 12, for use in the synthesis of oligonucletotides, peptides and carbohydrates by solid-phase synthesis technologies.

14. The multipurpose reaction vessel of claim 12, for use in the large scale purification of recombinant proteins, monoclonal antibodies and their conjugates, using affinity chromatography, ion exchange chromatography , or size-exclusion chromatography.

15. The multipurpose reaction vessel of claim 12 for use in solid and solution phase combinatorial synthesis of small molecules or the manufacture of small molecules.

16. The multipurpose reaction vessel of claim 12 for use in the preparation of functionalized solid matrices selected from beads, films and pins.

17. A reaction vessel for purification and isolation of molecules by chromatography comprising:
a cylindrical chamber comprising at least one solid/liquid/gas inlet and at least one solid/gas/liquid outlet;
a filter unit disposed in said cylindrical chamber capable of supporting chromatography material while allowing liquid phase reagents to pass below the filter;
a collection reservoir associated with the cylindrical chamber and in solid/liquid/gas contact with the cylindrical chamber below the filter; and
wherein the at least one solid/liquid/gas inlet and the at least one solid/liquid gas/ outlet are connected via a conduit means wherein said conduit means is connected to recycling means capable of causing the recycling of material between the at least one solid/liquid/gas inlet and the at least one solid/liquid gas outlet.

18. The reaction vessel of claim 17 wherein the chromatography is selected from affinity chromatography, ion exchange chromatography or size-exclusion chromatography.

19. The reaction vessel of claim 17 wherein the molecules are synthetic or biologically derived macromolecules.
